(19) United States
(12) Patent Application Publication
(10) Pub. No.: US 2024/0032819 A1
(43) Pub. Date: Feb. 1, 2024

(54) METHOD, APPARATUS AND SYSTEM FOR RECOGNIZING TREMOR SYMPTOM, RECOGNITION TERMINAL AND STORAGE MEDIUM

(71) Applicant: Regents of the University of Minnesota, Minneapolis, MN (US)

(72) Inventors: Qi Zhao, Minneapolis, MN (US); Ming Jiang, Minneapolis, MN (US); Xianyu Chen, Minneapolis, MN (US); Zhi Yang, Minneapolis, MN (US)

(21) Appl. No.: 18/448,995

(22) Filed: Aug. 14, 2023

(51) Int. Cl. A61B 5/11; A61B 5/00; G06V 10/82; G06V 10/20

(52) U.S. Cl. CPC A61B 5/1101; A61B 5/6898; G06V 10/82; G06V 10/20

(57) ABSTRACT

The present application relates to a method, apparatus and system for recognizing a tremor symptom, a recognition terminal and a storage medium. The method includes: receiving an image to be recognized which is uploaded by a user terminal, where the image to be recognized includes spiral graph used for recognizing whether a drawing person has a tremor state or not and evaluating a tremor level; and taking the image to be recognized as an input value of a pre-trained convolutional neural network regression device to obtain a tremor level. By using the method, the tremor can be recognized by means of images, which is non-invasive and patient-friendly, and since a model is a deep learning algorithm trained on a large spiral image dataset, results of evaluating the severity of essential tremor are accurate and consistent.

### TECHNICAL FIELD

The present application relates to the technical field of medical recognition, and in particular to a method, apparatus and system for recognizing a tremor symptom, a recognition terminal and a storage medium.

### BACKGROUND

Tremor is a symptom caused by neurological diseases such as Parkinson's disease, essential tremor and dystonia, which is characterized by involuntary rhythmic shaking of a certain part of the body. Tremor delivers a significant impact on a person's quality of life, affecting activities of daily living such as eating, drinking, writing, and even speaking. Accurate and objective quantification of the tremor symptom is essential for effective diagnosis, treatment, and recognition of illness.

Currently, methods for recognizing a tremor symptom include subjective rating scales, invasive electrophysiological techniques, and accelerometer-based devices. However, existing methods for recognizing a tremor symptom have limitations in terms of accuracy, convenience, and a patient experience feeling. For example, subjective rating scales rely on the patient's self-assessment, which may be influenced by anxiety, prejudice, or poor memory. Invasive electrophysiological techniques require electrodes to be inserted into the body, which may cause discomfort and risk of infection. Accelerometer-based devices measure tremor from motion of the devices, which may be affected by factors other than tremor, including, but not limited to, tremor, motion artifacts.

In summary, there is an urgent need for a method for recognizing a tremor symptom, which has high accuracy, high convenience, and a good patient experience feeling.

### SUMMARY

Based on this, it is necessary to provide a method, apparatus and system for recognizing a tremor symptom, which have high accuracy, high convenience, and a good patient experience feeling, a recognition terminal and a storage medium.

A method for recognizing a tremor symptom includes:
receiving an image to be recognized which is uploaded by a user terminal, where the image to be recognized includes a spiral graph used for recognizing whether a drawing person has a tremor state or not and evaluating a tremor level;
taking the image to be recognized as an input value of a pre-trained convolutional neural network regression device to obtain a tremor level; and
sending the tremor level to the user terminal.

In one example, the method further includes: training a set of images to be learned by means of a convolutional neural network model to obtain a convolutional neural network regression device, where all images to be learned in the set of images to be learned each include a spiral graph drawn by a patient.

In one example, the convolutional neural network model is based on a ResNet-18 backbone network, the ResNet-18 backbone network is composed of 18 parameterized layers, and the ResNet-18 backbone network includes a convolutional layer and a full connection layer, where an output layer of the full connection layer performs regression analysis to evaluate accuracy.

In one example, before the training a set of images to be learned by means of a convolutional neural network model to obtain a convolutional neural network regression device, the method further includes:
preprocessing each of the images to be learned in the set of images to be learned.

In one example, the preprocessing each of the images to be learned in the set of images to be learned specifically includes:
cropping each of the images to be learned, where each of the images to be learned subjected to cropping only retains a spiral graphic portion;
adjusting a size of each of the images to be learned subjected to cropping according to preset resolution;
normalizing each of the images to be learned subjected to size adjustment by means of histogram equalization or contrast stretching;
converting each of the images to be learned subjected to normalization into a grayscale image; and
augmenting each of the images to be learned subjected to grayscale, where the augmented manner includes: any one or a combination of more of random rotating, symmetrical flipping, scaling, perspective, changing brightness of images, contrast, saturation and hue, and inverting colors of given images.

In one example, the method further includes:
performing enhancement processing on each of the images to be learned and the image to be recognized.

In one example, the performing enhancement processing on each of the images to be learned and the image to be recognized specifically includes:
deblurring each of the images to be learned and the image to be recognized by using a non-blind deblurring algorithm, a Wiener filtering method or a bilateral filtering method; or/and
denoising each of the images to be learned and the image to be recognized by means of a median filter, an adaptive Wiener filter, a non-local self-similarity model, a sparse model, a gradient model, or a Markov random field model; or/and
performing contrast enhancement on each of the images to be learned and the image to be recognized by means of histogram equalization, histogram specification, contrast stretching, or local contrast enhancement.

In one example, the method further includes:
generating a report of a tremor level according to the tremor level and the historical tremor level associated with an ID of the user terminal.

An apparatus for recognizing a tremor symptom includes:
an image receiving module configured to receive an image to be recognized which is uploaded by a user terminal, where the image to be recognized includes a spiral graph used for recognizing whether a drawing person has a tremor state or not and evaluating a tremor level;
an image recognition module configured to take the image to be recognized as an input value of a pre-trained convolutional neural network regression device to obtain a tremor level; and
a result feedback module configured to send the tremor level to the user terminal.

A recognition terminal includes a memory and a processor, where the memory stores a computer program, and when the processor executes the computer program, the following steps are implemented:
receiving an image to be recognized which is uploaded by a user terminal, where the image to be recognized includes a spiral graph used for recognizing whether a drawing person has a tremor state or not and evaluating a tremor level;
taking the image to be recognized as an input value of a pre-trained convolutional neural network regression device to obtain a tremor level; and
sending the tremor level to the user terminal.

A computer-readable storage medium, on which a computer program is stored, and when the computer program is executed by a processor, the following steps are implemented:
receiving an image to be recognized which is uploaded by a user terminal, where the image to be recognized includes a spiral graph used for recognizing whether a drawing person has a tremor state or not and evaluating a tremor level;
taking the image to be recognized as an input value of a pre-trained convolutional neural network regression device to obtain a tremor level; and
sending the tremor level to the user terminal.

A system for recognizing a tremor symptom includes: a recognition terminal and at least one user terminal that execute the above method for recognizing a tremor symptom, where the recognition terminal and the at least one user terminal communicate by means of a network connection.

The recognition terminal is configured to train an image set to be learned by means of a convolutional neural network model to obtain a convolutional neural network regression device, where all images to be learned in the image set to be learned each include a spiral graph drawn by a patient.

The user terminal is configured to acquire the image to be recognized which includes a spiral graph, and send the image to be recognized to the recognition terminal.

The recognition terminal is further configured to receive an image to be recognized which is uploaded by the user terminal, where the image to be recognized includes a spiral graph used for recognizing whether a drawing person has a tremor state or not and evaluating a tremor level, is configured to take the image to be recognized as an input value of a pre-trained convolutional neural network regression device to obtain a tremor level, and is configured to send the tremor level to the user terminal.

The user terminal is further configured to receive and display the tremor level.

According to the above method, apparatus and system for recognizing a tremor symptom, the recognition terminal and the storage medium, the image to be recognized which is uploaded by the user terminal is received, the image to be recognized includes the spiral graph used for recognizing whether the drawing person has the tremor state or not and evaluating the tremor level, and the image to be recognized is taken as the input value of the pre-trained convolutional neural network regression device to obtain the tremor level. Compared with existing methods, the present application recognizes tremor by means of images, which is non-invasive and patient-friendly. Since the model is a deep learning algorithm trained on a large spiral image dataset, results of evaluating the severity of essential tremor are accurate and consistent.

### BRIEF DESCRIPTION OF THE DRAWINGS

FIG. 1 is a schematic structural diagram of a system for recognizing a tremor symptom in an example;

FIG. 2 is a schematic flowchart of a method for recognizing a tremor symptom in an example;

FIG. 3 is a schematic diagram of a linear relationship between a predicted tremor level evaluated value and a true level in another example;

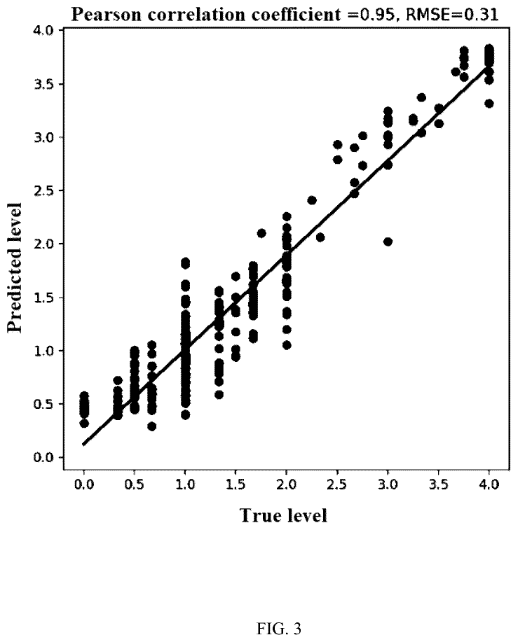

FIG. 4 is a schematic corresponding diagram of a spiral graph and a tremor level in an example;

FIG. 5 is a schematic diagram for a display effect of a tremor level in an example;

FIG. 6 is a schematic structural diagram of an apparatus for recognizing a tremor symptom in an example; and

FIG. 7 is an internal structural diagram of a recognition terminal in an example.

### DETAILED DESCRIPTION OF THE EMBODIMENTS

For making the objectives, technical solutions and advantages of the present application clearer, the present application will be described in further detail below in conjunction with the accompanying drawings and examples of the present application. It should be understood that the particular examples described herein are merely illustrative of the present application and are not intended to limit the present application.

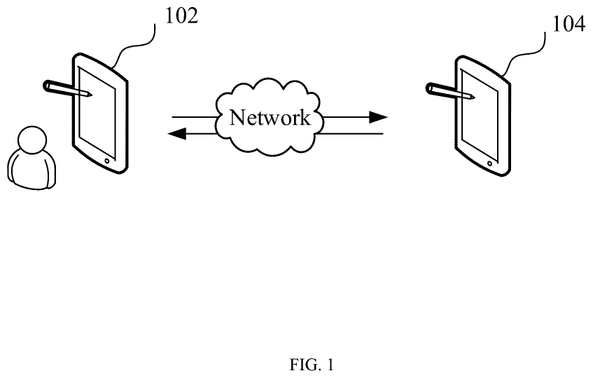

For a method for recognizing a tremor symptom provided by the present application, tremor in the present application specifically refers to essential tremor, which may be applied to an application environment as shown in FIG. 1, where a user terminal 102 communicates with a recognition terminal 104 by means of a network. The user terminal 102 includes, but not limited to, various personal computers, notebook computers, smart phones, tablet computers, and portable wearable devices, and the recognition terminal 104 includes, but not limited to, a mobile device, an independent server, or a server cluster composed of a plurality of servers. It should be noted that in a use scenario where the recognition terminal is equipped with a camera or equipped with a drawing receiving function, the user terminal and the recognition terminal are integrated into one device. The recognition terminal is configured to train an image set to be learned by means of a convolutional neural network model to obtain a convolutional neural network regression device, where all images to be learned in the image set to be learned each include a spiral graph drawn by a patient.

The user terminal is configured to acquire the image to be recognized which includes a spiral graph, and send the image to be recognized to the recognition terminal.

The recognition terminal is further configured to receive an image to be recognized which is uploaded by the user terminal, where the image to be recognized includes a spiral graph used for recognizing whether a drawing person has a tremor state or not and evaluating a tremor level, is configured to take the image to be recognized as an input value of a pre-trained convolutional neural network regression device to obtain a tremor level, and is configured to send the tremor level to the user terminal.

The user terminal is further configured to receive and display the tremor level.

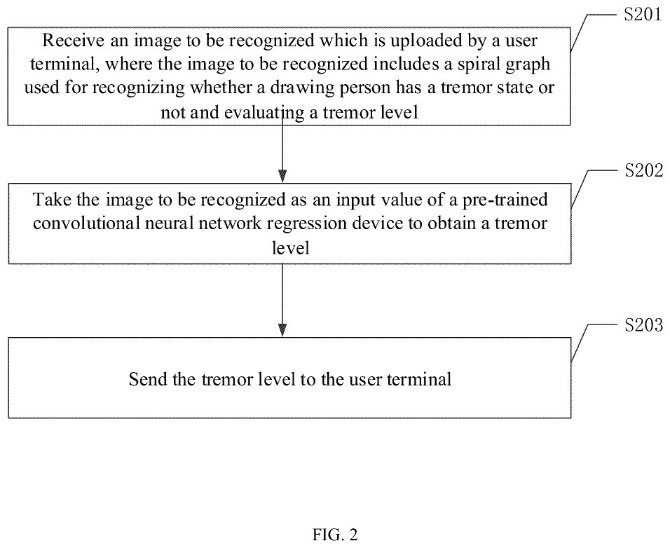

In an example, as shown in FIG. 2, a method for recognizing a tremor symptom is provided. Application of this method to a recognition terminal in FIG. 1 is taken as an example for illustration, and specifically, hand tremor is taken as an example for illustration, which includes the following steps:

S201, receive an image to be recognized which is uploaded by a user terminal, where the image to be recognized includes a spiral graph used for recognizing whether a drawing person has a tremor state or not and evaluating a tremor level.

In this example, the image to be recognized includes a spiral graph used for recognizing whether a drawing person has a tremor state or not and evaluating a tremor level, where the drawing person is a person who may have a tremor state. The drawing person may be instructed to use a pen or pencil for drawing completion on a piece of paper, and the drawer is instructed to draw a spiral from a center of the paper to an edge of the paper, which can be completed in about 30-60 seconds. An image to be recognized is acquired by photographing a spiral graph on the paper by means of a camera of the user terminal, and the image to be recognized is uploaded to the recognition terminal by the user terminal by means of the network. Or the image to be recognized can also be acquired by directly taking a picture by the recognition terminal equipped with a camera.

Preferably, before the step S202, the method further includes:
train a set of images to be learned by means of a convolutional neural network model to obtain a convolutional neural network regression device, where all images to be learned in the set of images to be learned each include a spiral graph drawn by a patient.

All the images to be learned in the set of images to be learned each include a spiral graph drawn by a patient, and the patient refers to a person who is diagnosed to have a tremor state. The convolutional neural network model is based on a ResNet-18 backbone network, and the ResNet-18 backbone network is composed of 18 parameterized layers and includes a convolutional layer and a full connection layer, where an output layer of the full connection layer performs regression analysis to evaluate accuracy, and the convolutional layer is responsible for extracting features from the input image. Specifically, the features of the input image include the shape, amplitude and frequency of the spiral graph, an activation layer introduces nonlinear changes into the network, a pooling layer is configured to reduce spatial resolution of the feature graph, and the full connection layer is configured to perform prediction according to the extracted features. Specifically, the output layer is adjusted from a predicted classification label to a single variable that predicts the severity of the essential tremor. The convolutional layer is slightly adjusted so as to recognize a pattern in the spiral graph, thereby accurately determining the severity of the tremor state. A modified architecture is trained on a large data set of the spiral graph to learn the mode of the hand tremor and quantify the severity thereof the hand tremor, thereby generating the convolutional neural network regression device, and taking the image including the spiral graph as the input value. The ResNet-18 architecture has the capability of learning complex features, and is very suitable for quantifying the severity of the essential tremor symptom from the spiral graph.

Specifically, regression analysis is performed by the fully connected output layer of the ResNet-18 architecture. The output layer is modified from the predicted classification label to a continuous value that predicts a predicted tremor level representing the image of the input spiral graph. Then, the predicted tremor level is compared to a true tremor level to evaluate the accuracy of the model. Regression analysis is a critical component of the convolutional neural network model because it enables the algorithm to quantize the severity of the essential tremor symptom on a continuous scale (rather than a classification scale). In this way, the severity of the essential tremor symptom can be evaluated more accurately, and more detailed information is provided for medical professionals, thereby making a judicious decision on a treatment scheme. Preferably, two indicators can be applied to evaluate internal test performance, and the indicators include a root-mean-square error (RMSE) and a Pearson correlation coefficient. The root-mean-square error is used for quantifying difference between the predicted tremor level and the true level, and a method for calculating the indicator is to average the difference between the predicted level and the true level of each image, and then take the root. The lower the root-mean-square error is, the closer the predicted level is to the true level, indicating that the higher the accuracy of the model is. In addition, the Pearson correlation coefficient is also calculated to evaluate a linear relationship between the predicted tremor level and the true level, a value range of the indicator is −1-1, where 1 represents a strong positive correlation, −1 represents a strong negative correlation, and 0 represents an irrelevant condition. The higher Pearson correlation coefficient indicates the predicted tremor level is highly related to the true level. The combination of the root-mean-square error and the Pearson correlation coefficient provides a comprehensive evaluation of the internal test performance. During internal testing, the two indicators of a test data set are calculated and used as measurement criteria for the algorithm accuracy. As a result, the algorithm has a lower root-mean-square error (0.31) and a higher Pearson correlation coefficient (0.95), indicating that the algorithm has good performance in predicting the tremor level of the image of the spiral graph.

S202, take the image to be recognized as an input value of a pre-trained convolutional neural network regression device to obtain a tremor level.

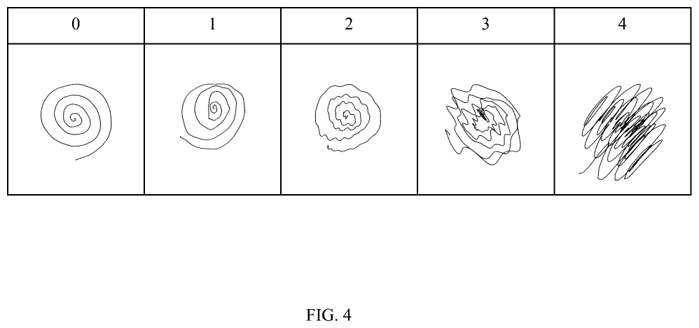

In this example, the set of images to be learned is trained by the convolutional neural network model to obtain the pre-trained convolutional neural network regression device, the image to be recognized is taken as the input value of the pre-trained convolutional neural network regression device, and a continuous output value of the convolutional neural network regression device is converted into a classified tremor level. The tremor level represents the severity of the essential tremor, and specifically the level is represents by means of the digitals of 0-4, where level 0 represents no tremor, level 4 represents the most serious tremor, and the larger the level value from 1 to 4 is, the more serious the tremor is. As shown in FIG. 4, different tremor severity levels correspond to different spiral graphs.

S203, send the tremor level to the user terminal.

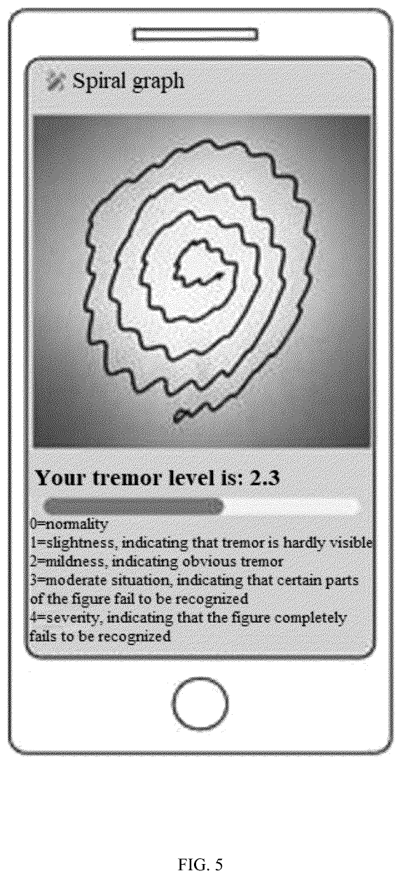

In this example, the recognition terminal sends the tremor level to the user terminal, and at the same time, saves information such as the tremor level and a user ID associated with the corresponding user terminal into a database, and the user terminal displays the tremor level, as shown in FIG. 5.

Preferably, after the step S203, the method further includes: generate a report of a tremor level according to the tremor level and the historical tremor level associated with the ID of the user terminal. At the same time, the tremor level is saved in the database, such that a medical worker can track changes of the tremor severity over time.

In the above method for recognizing a tremor symptom, the image to be recognized which is uploaded by the user terminal is received, the image to be recognized includes the spiral graph used for recognizing whether the drawing person has the tremor state or not and evaluating the tremor level, and the image to be recognized is taken as the input value of the pre-trained convolutional neural network regression device to obtain the tremor level. Compared with existing methods, the present application recognizes tremor by means of images, which is non-invasive and patient-friendly. Since the model is a deep learning algorithm trained on a large spiral image dataset, results of evaluating the severity of essential tremor are accurate and consistent.

In an example, the method further includes:
preprocess each of the images to be learned in the set of images to be learned.

Specifically, the step of preprocessing each of the images to be learned in the set of images to be learned specifically includes:
crop each of the images to be learned, where each of the images to be learned subjected to cropping only retains a spiral graphic portion;
adjust a size of each of the images to be learned subjected to cropping according to preset resolution;
normalize each of the images to be learned subjected to size adjustment by means of histogram equalization or contrast stretching;
convert each of the images to be learned subjected to normalization into a grayscale image; and
augment each of the images to be learned subjected to grayscale, where the augmented manner includes: any one or a combination of more of random rotating, symmetrical flipping, scaling, perspective, changing brightness of images, contrast, saturation and hue, and inverting colors of given images.

According to the example of the present invention, the preprocessing step is crucial for ensuring the consistency of the input images and accurately reflecting the tremor severity of an individual, and the model itself can also more accurately evaluate the tremor severity and provide reliable results by normalizing the input images and expanding the scale of the training data set.

In an example, the method further includes:
perform enhancement processing on each of the images to be learned and the image to be recognized.

Specifically, the step of performing enhancement processing on each of the images to be learned and the image to be recognized specifically includes:
deblur each of the images to be learned and the image to be recognized by using a non-blind deblurring algorithm, a Wiener filtering method or bilateral filtering method; or/and
denoise each of the images to be learned and the image to be recognized by means of a median filter, an adaptive Wiener filter, a non-local self-similarity model, a sparse model, a gradient model, or a Markov random field model; or/and
perform contrast enhancement on each of the images to be learned and the image to be recognized by means of histogram equalization, histogram specification, contrast stretching, or local contrast enhancement.

According to the example of the present invention, image enhancement is combined with the above preprocessing step for use, the quality and accuracy of input data of the deep learning model can be improved, and the model can more accurately evaluate the severity of tremor and provide reliable results for the patient with essential tremor by reducing blurring caused by tremor and other image quality influence.

It should be understood that although the various steps in the flowchart of FIG. 2 are shown sequentially as indicated by the arrows, these steps are not necessarily performed sequentially in the order indicated by the arrows. Unless explicitly stated herein, these steps are not strictly limited to the order in which they are performed, and these steps may be performed in other orders. Moreover, at least part of the steps in FIG. 2 may include multiple sub-steps or multiple stages, and these sub-steps or stages are not necessarily executed at the same time, but may be executed at different times, and the execution sequence of these sub-steps or stages is not necessarily performed sequentially, but may be executed in turn or alternatively with other steps or at least part of sub-steps or stages of other steps.

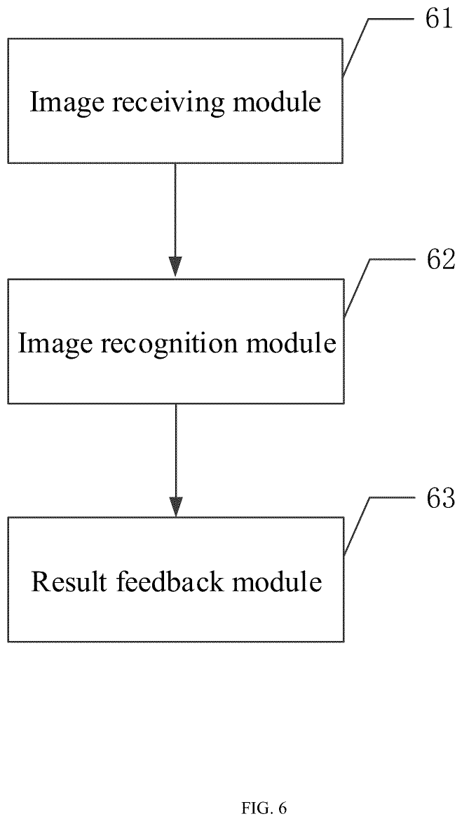

In an example, as shown in FIG. 6, an apparatus for recognizing a tremor symptom is provided. The apparatus includes an image receiving module 61, an image recognition module 62, and a result feedback module 63.

The image receiving module 61 is configured to receive an image to be recognized which is uploaded by a user terminal, and the image to be recognized includes a spiral graph used for recognizing whether a drawing person has a tremor state or not and evaluating a tremor level.

The image recognition module 62 is configured to take the image to be recognized as an input value of a pre-trained convolutional neural network regression device to obtain a tremor level.

The result feedback module 63 is configured to send the tremor level to the user terminal.

Furthermore, the apparatus further includes:
an image learning module which is configured to train a set of images to be learned by means of a convolutional neural network model to obtain a convolutional neural network regression device, where all images to be learned in the set of images to be learned each include a spiral graph drawn by a patient.

Preferably, the convolutional neural network model is based on a ResNet-18 backbone network, the ResNet-18 backbone network is composed of 18 parameterized layers, and includes a convolutional layer and a full connection layer, where an output layer of the full connection layer performs regression analysis to evaluate accuracy.

Furthermore, the apparatus further includes:
an image preprocessing module which is configured to preprocess each of the images to be learned in the set of images to be learned.

Specifically, the image preprocessing module is specifically configured to crop each of the images to be learned, where each of the images to be learned subjected to cropping only retains a spiral graphic portion; to adjust a size of each of the images to be learned subjected to cropping according to preset resolution; to normalize each of the images to be learned subjected to size adjustment by means of histogram equalization or contrast stretching; to convert each of the images to be learned subjected to normalization into a grayscale image; and to augment each of the images to be learned subjected to grayscale, where the augmented manner includes: any one or a combination of more of random rotating, symmetrical flipping, scaling, perspective, changing brightness of images, contrast, saturation and hue, and inverting colors of given images.

Furthermore, the apparatus further includes:
an image enhancement module which is configured to perform enhancement processing on each of the images to be learned and the image to be recognized.

Specifically, the image enhancement module is configured to deblur each of the images to be learned and the image to be recognized by using a non-blind deblurring algorithm, a Wiener filtering method or a bilateral filtering method; or/and denoise each of the images to be learned and the image to be recognized by means of a median filter, an adaptive Wiener filter, a non-local self-similarity model, a sparse model, a gradient model, or a Markov random field model; or/and
perform contrast enhancement on each of the images to be learned and the image to be recognized by means of histogram equalization, histogram specification, contrast stretching, or local contrast enhancement.

Furthermore, the apparatus further includes:
a report generation module which is configured to generate a report of a tremor level according to the tremor level and the historical tremor level associated with an ID of the user terminal.

For the specific definition of the apparatus for recognizing a tremor symptom, reference may be made to the definition of the method for recognizing a tremor symptom above, which is not repeated herein. Each module in the above apparatus for recognizing a tremor symptom may be implemented wholly or partially by means of software, hardware, or a combination thereof. The above modules may be embedded in or independent of a processor in a recognition terminal in a hardware form, or may be stored in a memory in the recognition terminal in a software form, such that the processor may call and execute operations corresponding to the above modules.

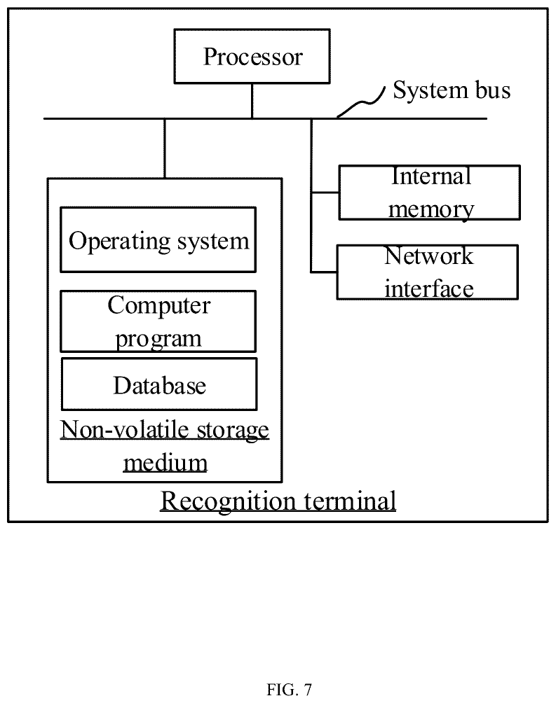

In an example, a recognition terminal is provided, and the recognition terminal may be a server, and an internal structural diagram thereof may be as shown in FIG. 7. The recognition terminal includes a processor, a memory, a network interface and a database that are connected by means of a system bus. The processor of the recognition terminal is configured to provide computing and control capabilities. The memory of the recognition terminal includes a non-volatile storage medium and an internal memory. The non-volatile storage medium stores an operating system, a computer program, and a database. The internal memory provides an environment for running of the operating system and the computer program in the non-volatile storage medium. The database of the recognition terminal is configured to store data. The network interface of the recognition terminal is configured to communicate with an external terminal via a network connection. The computer program, when executed by the processor, implements a method for recognizing a tremor symptom.

In an example, a recognition terminal is provided, the recognition terminal may be a mobile terminal, and an internal structural diagram thereof may be as shown in FIG. 7. The recognition terminal includes a processor, a memory, a network interface, a display screen and an input apparatus that are connected by means of a system bus. The processor of the recognition terminal is configured to provide computing and control capabilities. The memory of the recognition terminal includes a non-volatile storage medium and an internal memory. The non-volatile storage medium stores an operating system and a computer program. The internal memory provides an environment for running of the operating system and the computer program in the non-volatile storage medium. The network interface of the recognition terminal is configured to communicate with an external terminal via a network connection. The computer program, when executed by the processor, implements a method for recognizing a tremor symptom. The display screen of the recognition terminal may be a liquid crystal display screen or an electronic ink display screen, and the input apparatus of the recognition terminal may be a touch layer covered on the display screen, may also be a key, a trackball or a touch pad arranged on a housing of the recognition terminal, and may also be an external keyboard, a touch pad or a mouse, etc.

Those skilled in the art may understand that the structure shown in FIG. 7 is only a block diagram of part of the structure related to the solution of the present application, and does not constitute a limitation on the recognition terminal to which the solution of the present application is applied. A specific recognition terminal may include more or less components than those shown in the figure, or combine some components, or have a different component arrangement.

In an example, a recognition terminal is provided and includes a memory and a processor, where the memory stores a computer program, and when executing the computer program, the processor implements the following steps:
receive an image to be recognized which is uploaded by a user terminal, where the image to be recognized includes a spiral graph used for recognizing whether a drawing person has a tremor state or not and evaluating a tremor level;
take the image to be recognized as an input value of a pre-trained convolutional neural network regression device to obtain a tremor level; and
send the tremor level to the user terminal.

In an example, a computer-readable storage medium is provided, a computer program is stored on the storage medium, and when the computer program is executed by a processor, the following steps are implemented:
receive an image to be recognized which is uploaded by a user terminal, where the image to be recognized includes a spiral graph used for recognizing whether a drawing person has a tremor state or not and evaluating a tremor level;
take the image to be recognized as an input value of a pre-trained convolutional neural network regression device to obtain a tremor level; and
send the tremor level to the user terminal.

Those skilled in the art may understand that implementation of all or some procedures in the methods of the above examples may be accomplished by instructing related hardware by means of a computer program. The computer program may be stored in a non-volatile computer-readable storage medium, and when the computer program is executed, the procedures of the examples in the above methods may be included. Any reference to a memory, storage, a database, or other media used in the examples provided by the present application may include non-volatile and/or volatile memory. The non-volatile memory may include a read only memory (ROM), a programmable ROM (PROM), an electrically programmable ROM (EPROM), an electrically erasable programmable ROM (EEPROM), or a flash memory. The volatile memory may include a random access memory (RAM) or an external cache memory. By way of illustration and not limitation, RAM is available in many forms such as a static RAM (SRAM), a dynamic RAM (DRAM), a synchronous DRAM (SDRAM), a double data rate SDRAM (DDRSDRAM), an enhanced SDRAM (ESDRAM), a Synchlink DRAM (SLDRAM), a Rambus direct RAM (RDRAM), a direct Rambus dynamic RAM (DRDRAM), and a Rambus dynamic RAM (RDRAM), etc.

Various technical features of the embodiments mentioned above may be arbitrarily combined. To simplify description, all possible combinations of the various features of the embodiments mentioned above are not described. However, if only the combinations of these technical features do not conflict, they shall be considered to be within the scope of description of the present invention.

The embodiments mentioned above are merely several embodiments of the present application, and are specifically described in details, but cannot be interpreted as limiting the scope of the patent for the invention as a result. It shall be noted that for those of ordinary skill in the field, they may make several transformations and improvements on the premise of not deviating from the conception of the present application, and these transformations and improvements shall fall within the scope of protection of the application. Hence, the scope of protection of the patent for the present application shall be subject to the appended claims.

1. A method for recognizing a tremor symptom, comprising:
receiving an image to be recognized which is uploaded by a user terminal, wherein the image to be recognized comprises a spiral graph used for recognizing whether a drawing person has a tremor state or not and evaluating a tremor level;
taking the image to be recognized as an input value of a pre-trained convolutional neural network regression device to obtain a tremor level; and
sending the tremor level to the user terminal.

2. The method according to claim 1, wherein before the receiving an image to be recognized which is uploaded by a user terminal, the method further comprises:
training a set of images to be learned by means of a convolutional neural network model to obtain a convolutional neural network regression device, wherein all images to be learned in the set of images to be learned each comprise a spiral graph drawn by a patient.

3. The method according to claim 1, wherein the convolutional neural network model is based on a ResNet-18 backbone network, the ResNet-18 backbone network is composed of 18 parameterized layers, and the ResNet-18 backbone network comprises a convolutional layer and a full connection layer, wherein an output layer of the full connection layer performs regression analysis to evaluate accuracy.

4. The method according to claim 1, wherein before the training a set of images to be learned by means of a convolutional neural network model to obtain a convolutional neural network regression device, the method further comprises:
preprocessing each of the images to be learned in the set of images to be learned.

5. The method according to claim 4, wherein the preprocessing each of the images to be learned in the set of images to be learned specifically comprises:
cropping each of the images to be learned, wherein each of the images to be learned subjected to cropping only retains a spiral graphic portion;
adjusting a size of each of the images to be learned subjected to cropping according to preset resolution;
normalizing each of the images to be learned subjected to size adjustment by means of histogram equalization or contrast stretching;
converting each of the images to be learned subjected to normalization into a grayscale image; and
augmenting each of the images to be learned subjected to grayscale, wherein the augmented manner comprises: any one or a combination of more of random rotating, symmetrical flipping, scaling, perspective, changing brightness of images, contrast, saturation and hue, and inverting colors of given images.

6. The method according to claim 4, wherein after the preprocessing each of the images to be learned in the set of images to be learned, the method further comprises:
performing enhancement processing on each of the images to be learned and the image to be recognized.

7. The method according to claim 6, wherein the performing enhancement processing on each of the images to be learned and the image to be recognized specifically comprises:
deblurring each of the images to be learned and the image to be recognized by using a non-blind deblurring algorithm, a Wiener filtering method or a bilateral filtering method; or/and
denoising each of the images to be learned and the image to be recognized by means of a median filter, an adaptive Wiener filter, a non-local self-similarity model, a sparse model, a gradient model, or a Markov random field model; or/and
performing contrast enhancement on each of the images to be learned and the image to be recognized by means of histogram equalization, histogram specification, contrast stretching, or local contrast enhancement.

8. The method according to claim 1, further comprises:
generating a report of a tremor level according to the tremor level and the historical tremor level associated with an ID of the user terminal.

9. An apparatus for recognizing a tremor symptom, comprising:
an image receiving module configured to receive an image to be recognized which is uploaded by a user terminal, wherein the image to be recognized comprises a spiral graph used for recognizing whether a drawing person has a tremor state or not and evaluating a tremor level;
an image recognition module configured to take the image to be recognized as an input value of a pre-trained convolutional neural network regression device to obtain a tremor level; and
a result feedback module configured to send the tremor level to the user terminal.

10. A recognition terminal, comprising a memory and a processor, wherein the memory stores a computer program, and when the processor executes the computer program, the steps of the method according to claim 1 are implemented.

11. A computer-readable storage medium, on which a computer program is stored, and when the computer program is executed by a processor, the steps of the method according to claim 1 are implemented.

12. A system for recognizing a tremor symptom, comprising: a recognition terminal and at least one user terminal that execute the method for recognizing a tremor symptom according to claim 1, wherein the recognition terminal and the at least one user terminal communicate by means of a network connection,
the recognition terminal is configured to train an image set to be learned by means of a convolutional neural network model to obtain a convolutional neural network regression device, wherein all images to be learned in the image set to be learned each comprise a spiral graph drawn by a patient;
the user terminal is configured to acquire the image to be recognized which comprises a spiral graph, and send the image to be recognized to the recognition terminal;
the recognition terminal is further configured to receive an image to be recognized which is uploaded by the user terminal, wherein the image to be recognized comprises a spiral graph used for recognizing whether a drawing person has a tremor state or not and evaluating a tremor level, is configured to take the image to be recognized as an input value of a pre-trained convolutional neural network regression device to obtain a tremor level, and is configured to send the tremor level to the user terminal; and
the user terminal is further configured to receive and display the tremor level.
